(19) United States
(12) Patent Application Publication
(10) Pub. No.: US 2016/0287811 A1
(43) Pub. Date: Oct. 6, 2016

(54) INJECTION DEVICES AND METHODS OF USE THEREOF

(71) Applicant: Boston Scientific Scimed, Inc., Maple Grove, MN (US)

(72) Inventors: Elisabeth GABIN, Dracut, MA (US); Sharon HUNTER, Holliston, MA (US); Thomas JONES, Franklin, MA (US); Gary LEANNA, Holden, MA (US); Eric SCHNEIDER, Cumberland, RI (US); Manoj MADHAVAN, Woolahra (AU); Benedicta CHUMO, Framingham, MA (US)

(73) Assignee: Boston Scientific Scimed, Inc.

(21) Appl. No.: 15/082,147

(22) Filed: Mar. 28, 2016

Related U.S. Application Data
(60) Provisional application No. 62/142,607, filed on Apr. 3, 2015.

(51) Int. Cl. A61M 5/32

(52) U.S. Cl. CPC A61M 5/3297; A61M 5/3202; A61M 5/3243

(57) ABSTRACT

Medical devices for injecting tissue and methods of use thereof are described. The medical device may comprise a handle, a shaft, and a plurality of needles extending through the shaft, each needle defining a lumen therethrough and being independently moveable along a longitudinal axis of the device to transition from a retracted configuration to an extended configuration. In the extended configuration, a distal portion of each needle may deflect radially outward with respect to the longitudinal axis of the device. The medical device may comprise an end cap coupled to the distal end of the shaft and/or a locking mechanism to selectively lock the needles in the retracted configuration and/or extended configuration.

### CROSS-REFERENCE TO RELATED APPLICATION(S)

This application claims benefit of priority under U.S.C. §119 to U.S. Provisional Patent Application No. 62/142,607, filed Apr. 3, 2015, the entirety of which is incorporated herein by reference.

### TECHNICAL FIELD

Embodiments of the present disclosure relate generally to medical devices and related methods of use. More specifically, the present disclosure relates to injection devices and methods of injecting fluid into patient tissue.

### BACKGROUND

Abnormal tissue growth can inhibit normal passage of fluids and other materials through the body. For example, tumors along the esophagus (e.g., cancerous tissue resulting from esophageal cancer, or benign tissue masses associated with other health conditions) can lead to dysphagia, and can inhibit the passage of food and liquids. Without treatment, such tissue overgrowth can pose serious health concerns and impair a patient's quality of life. In some cases, it may be desirable to seek treatments for palliative care, e.g., to alleviate the distress of the tissue blockage.

### SUMMARY OF THE DISCLOSURE

The present disclosure includes a medical device comprising a handle including a plurality of actuators; a shaft; and a plurality of needles extending through the shaft, each needle defining a lumen, and each needle corresponding to one of the plurality of actuators for movement along a longitudinal axis of the device from a retracted configuration to an extended configuration, independent of movement of the other needles of the plurality of needles; wherein, in the extended configuration, a distal portion of at least one of the needles deflects radially outward with respect to the longitudinal axis of the device. According to some aspects, a distal portion of each needle may deflect radially outward with respect to the longitudinal axis of the device. According to some aspects, each actuator may extend through a corresponding slot of the handle.

The distal portion of each needle may include a sharp tip configured to puncture tissue. Additionally or alternatively, the distal portion of at least one of the needles may include a plurality of apertures disposed circumferentially around a wall of the needle. Further, a proximal portion of each needle optionally may include an attachment for coupling the needle to a fluid reservoir. For example, the fluid reservoir may comprise a syringe. According to some aspects, the device may include an actuator for controlling a supply of fluid to at least one of the needles, which may be separate from the actuators that move the needles along the longitudinal axis of the device.

According to some aspects, the medical device may comprise an end cap coupled to a distal end of the shaft, the end cap including at least one opening for the passage of one or more of the needles distally through the end cap. The end cap may include a plurality of openings arranged circumferentially about the end cap at a same radial position between a proximal end of the end cap and a distal end of the end cap. According to some aspects, the end cap may define a lumen, e.g., in communication with each opening of the plurality of openings. The end cap may be permanently attached or detachable from the shaft. Additionally or alternatively, the end cap may have a flared shape. According to some aspects, the medical device may include a feature to center the device within a lumen, e.g., upon inserting the medical device into the lumen. Additionally or alternatively, the medical device may comprise a locking mechanism corresponding to each needle of the plurality of needles to selectively lock the corresponding needle in at least one of the retracted configuration or the extended configuration. For example, each locking mechanism may include complementary mating elements of the needle and the handle, such that engaging the mating elements inhibits movement of the needle relative to the handle.

The present disclosure further includes a medical device comprising a handle including a plurality of actuators; a shaft; an end cap coupled to a distal end of the shaft; and a plurality of needles extending through the shaft and at least partially through the end cap, each needle defining a lumen and being independently moveable relative to other needles of the plurality of needles along a longitudinal axis of the device to transition from a retracted configuration to an extended configuration; wherein, in the extended configuration, a distal portion of at least one of the needles (e.g., a distal portion of each needle) deflects radially outward with respect to the longitudinal axis of the device.

According to some aspects, the end cap may include a plurality of openings arranged circumferentially about the end cap at a same radial position between a proximal end of the end cap and a distal end of the end cap, each needle being aligned with one of the openings. Additionally or alternatively, the end cap may be detachable from the shaft. For example, the end cap may include at least one mating element complementary to a mating element of the shaft for coupling the end cap to the shaft, and detaching the end cap from the shaft.

The present disclosure further includes a medical device comprising a handle; a shaft; an end cap coupled to a distal end of the shaft; a plurality of needles extending through each of the shaft and the end cap, each needle defining a lumen and being independently moveable relative to other needles of the plurality of needles along a longitudinal axis of the device to transition from a retracted configuration to an extended configuration; and a locking mechanism corresponding to each needle of the plurality of needles to selectively lock the corresponding needle in at least one of the retracted configuration or the extended configuration.

According to some aspects, when each needle is in the extended configuration, a distal portion of the needle may deflect radially outward with respect to the longitudinal axis of the device. Additionally or alternatively, the handle may include a plurality of actuators, each actuator configured to move a corresponding one of the needles along the longitudinal axis of the device independently of the other needles.

### BRIEF DESCRIPTION OF THE FIGURES

The accompanying drawings, which are incorporated in and constitute a part of this specification, illustrate various exemplary embodiments and together with the description, serve to explain the principles of the disclosed embodiments.

FIG. 1 shows an exemplary medical device positioned in a body lumen according to some aspects of the present disclosure.

FIG. 2 shows an exemplary medical device according to some aspects of the present disclosure.

FIGS. 3A and 3B show an exemplary end cap according to some aspects of the present disclosure.

FIGS. 4A and 4B show another exemplary end cap according to some aspects of the present disclosure.

FIGS. 5A, 5B, and 5C illustrate various mechanisms for coupling an end cap to a shaft according to some aspects of the present disclosure.

FIG. 6 shows an exemplary medical device according to some aspects of the present disclosure.

FIG. 7 shows a handle portion of an exemplary medical device according to some aspects of the present disclosure.

### DETAILED DESCRIPTION

Embodiments of the present disclosure include medical devices useful for injecting a fluid into patient tissue. The injection may form part of a treatment session or regime intended to decrease tissue mass and/or to prevent or inhibit tissue growth. For example, a fluid such as ethanol (or other alcohol or alcoholic mixture) may be injected into a mass of tissue causing partial or total blockage of a body channel, in order to alleviate the blockage by shrinking the stricture. Without being bound by theory, it is believed that alcoholization may cause tumor coagulation, tissue necrosis through cellular dehydration, protein denaturation, fibrosis, and/or microvascular thrombosis.

For example, the present disclosure may be useful to treat dysphagia by reducing abnormal tissue growth in the esophagus. The devices and methods disclosed herein also may be useful for cytokine and/or chemotherapy treatments. While certain aspects of the following description are illustrated within the context of treating the esophagus, it is understood that the present disclosure is not limited to a particular part of the body or health condition, and may have multiple applications, consistent with the principles herein.

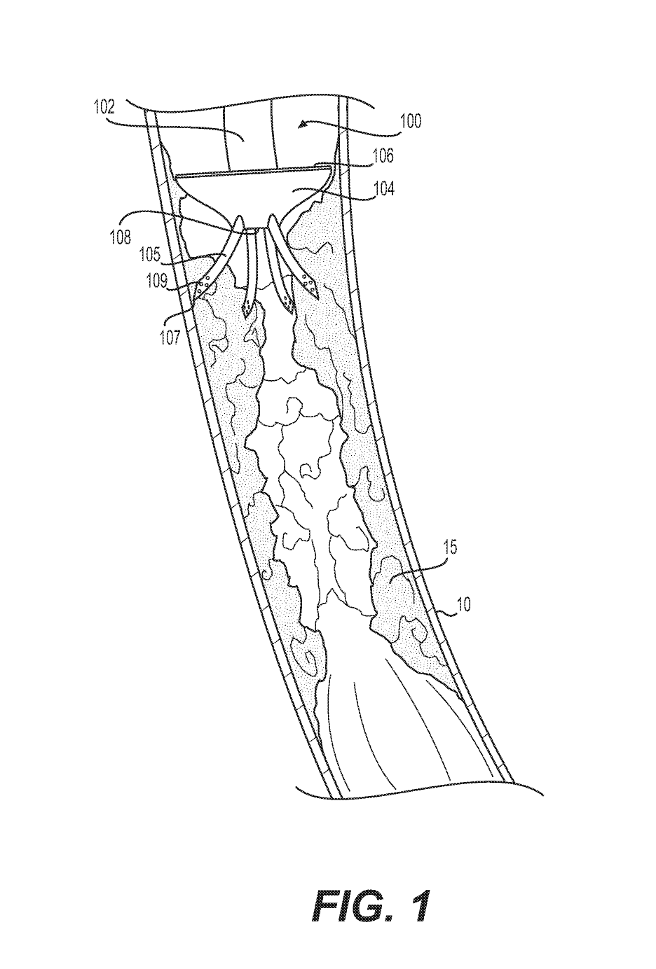

FIG. 1 shows a body lumen 10, e.g., the esophagus, with abnormal or excess tissue 15 along the wall of the lumen 10. The excess tissue 15 may cause strictures that narrow the lumen diameter and inhibit the passage of materials therethrough. For example, strictures along the esophageal wall in FIG. 1 may inhibit the ability to eat, drink, and/or swallow due to the reduced inner diameter of the lumen 10. The excess tissue 15 may be malignant tissue resulting from cancer, for example, or benign tissue associated with another health condition. A medical device 100 may be inserted into the esophagus 10 proximate the strictures and used to inject a fluid, such as an alcohol, into the excess tissue 15. As mentioned above, the alcohol may reduce the stricture and facilitate the passage of food and liquids through the esophagus 10.

The medical device 100 may include at least one needle 105 extending through a shaft 102 of the device 100 to exit through an end cap 104 of the device 100. The term “end cap” is used herein for convenience in referring to the figures, and does not exclude devices according to the present disclosure that may have features distal to the end cap. In some embodiments, for example, the device 100 may include a plurality of needles 105 as shown in FIG. 1. Medical devices according to the present disclosure may include any suitable number of needles 105, e.g., 2, 3, 4, 5, or 6 or more needles 105. The needles 105 may be disposed within a single lumen of the shaft 102, or the shaft 102 may include separate lumens to separate a needle 105 from one or more other needles 105. At least one of the needles 105 may deflect radially outward past the distal end of the end cap 104 for contacting the tissue 15 at different radial positions around the circumference of the lumen 10. In addition or alternatively, at least one of the needles 105 may exit the distal end of the end cap 104 without deflecting, e.g., extending along, or parallel to, the longitudinal axis of the device 100. According to some aspects of the present disclosure, a plurality of the needles 105, e.g., all of the needles 105, may deflect radially outward, at different radial positions. The device 100 may be translatable and/or rotatable within the lumen 10 to allow the needles 105 to contact different portions of the tissue 15.

Each needle 105 may include a sharp distal tip 107 for puncturing the tissue 15. The needles 105 may include one or more apertures for injecting the fluid, e.g., a distal-facing aperture defined by the tip 107 and/or one or more side apertures 109 proximate the tip 107, e.g., along the wall of the needle 105. In some embodiments, the needles 105 may include a plurality of side apertures 109 disposed circumferentially around the needle 105 proximate the tip 107, e.g., for injecting the fluid into the tissue 15 in an evenly distributed manner.

The device 100 may be used with or without an endoscope or other access device. In some embodiments, the end cap 104 may have dimensions suitable for centering the device 100 in a body lumen, e.g., for use without an endoscope, wherein an outermost dimension of the end cap 104 may be approximately the same as the diameter of the body lumen 10. The end cap 104 may have any suitable shape. According to some aspects, for example, the end cap 104 may have a flared profile, e.g., having an outer diameter increasing from distal end to proximal end or vice versa. As shown in FIG. 1, the end cap 104 may have a circular cross-section, wherein the outermost diameter may be approximately equal to, or slightly smaller than, the inner diameter of the esophagus 10 (e.g., the diameter of the esophagus 10 proximate the stricture location). The end cap 104 may have a generally frustoconical and atraumatic shape, the proximal end 106 having a greater diameter than the distal end 108. The largest cross-sectional dimension of the end cap 104 need not be at the proximal end 106, however, and may be at the distal end 108 or between the proximal and distal ends 106, 108. In some embodiments, the device 100 may be configured for insertion into an endoscope, e.g., wherein the end cap 104 may have a substantially constant cross-sectional dimension from proximal end 106 to distal end 108.

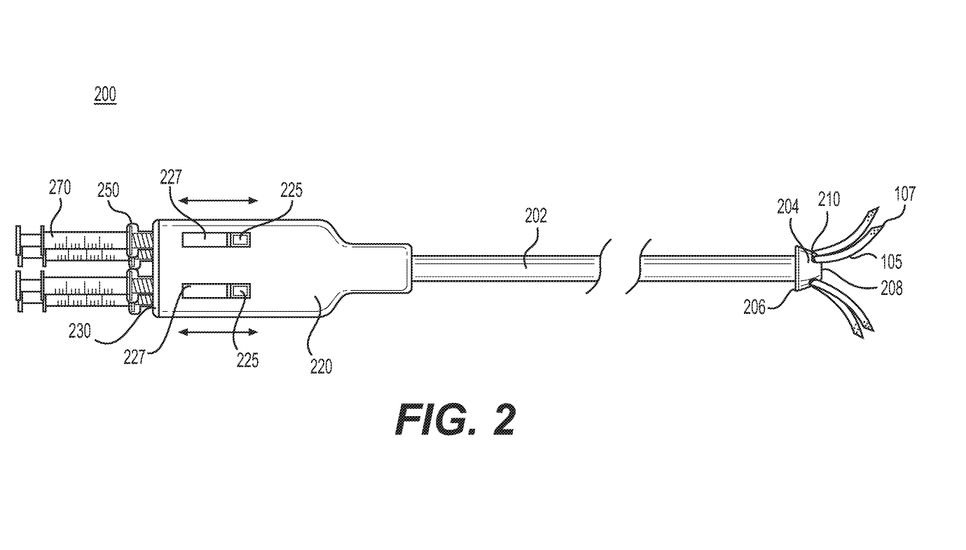

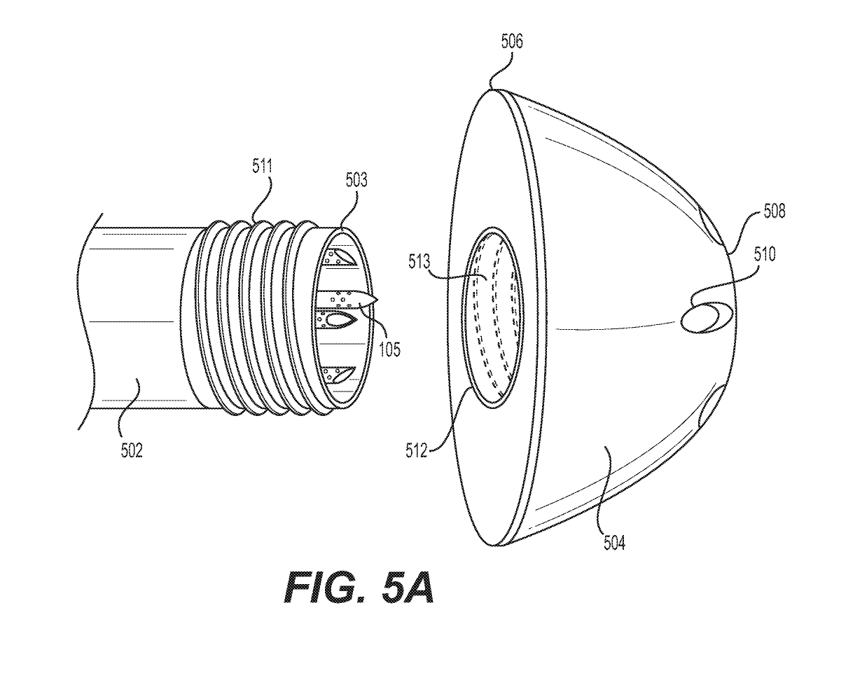

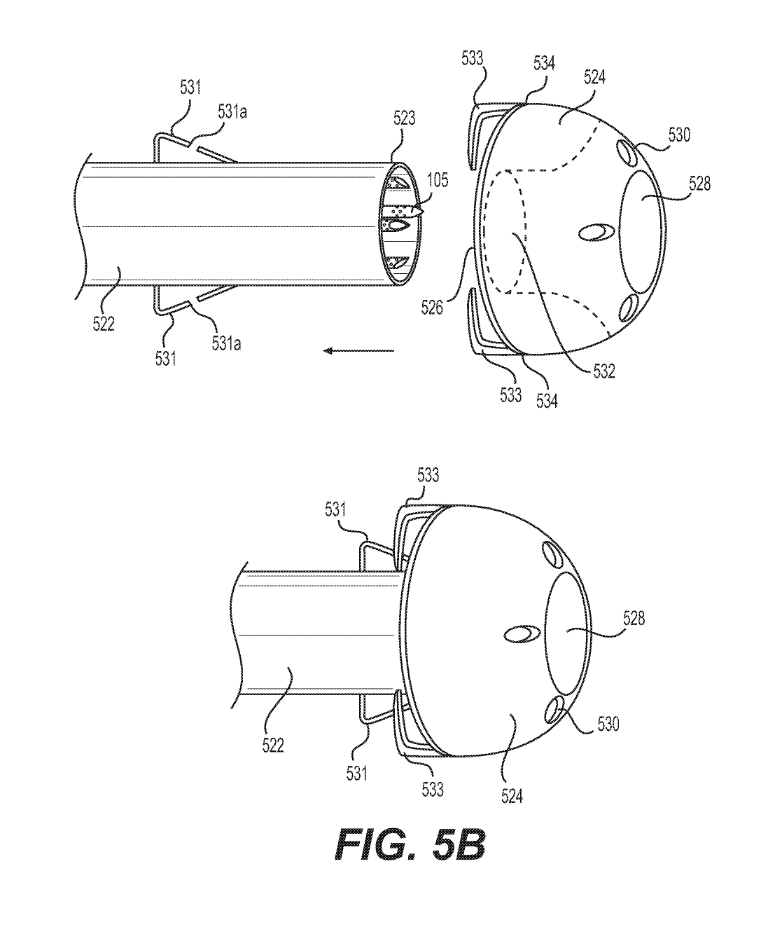

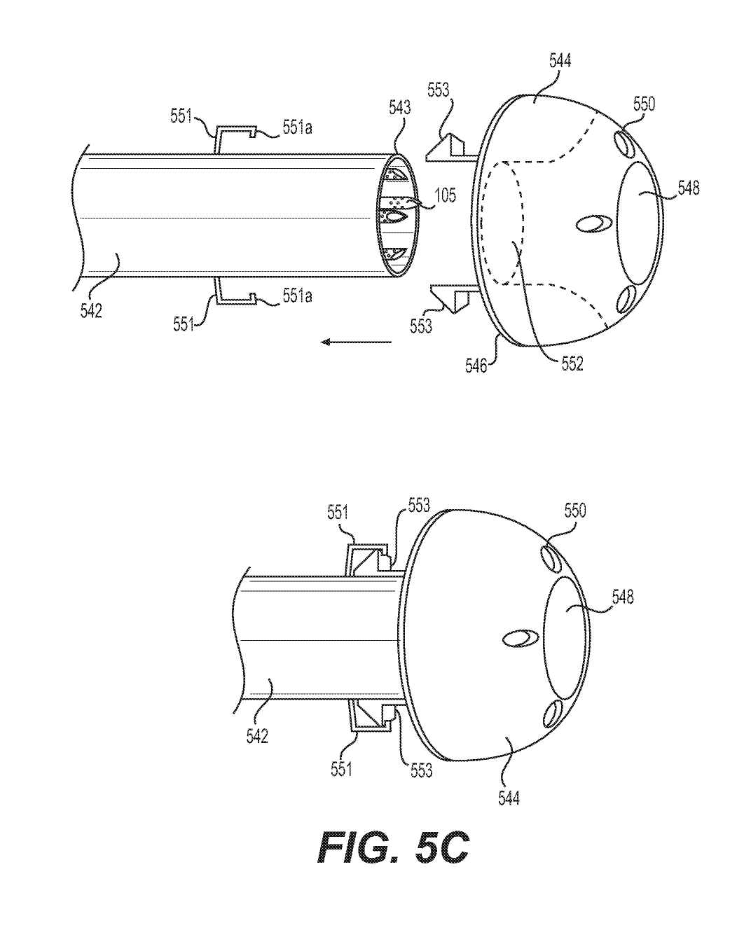

FIG. 2 illustrates another exemplary medical device 200 for deploying the needles 105, which may include any of the features of device 100. As shown, the device 200 includes a handle 220, a shaft 202, and an end cap 204. The end cap 204 may be coupled to the distal end of the shaft 202 via a suitable mechanism (see, e.g., FIGS. 5A-5C, discussed below). The end cap 204 and the shaft 202 may be permanently attached (e.g., via an adhesive) or detachable from each other. In some embodiments, the end cap 204 may be integral with the shaft 202 such that they form a single, integral component. The shaft 202 may comprise a single lumen (e.g., all needles 105 disposed within the same lumen) or plurality of lumens (e.g., a separate lumen to receive each needle 105). The shaft 202 may be flexible, and may comprise a material with sufficient strength to protect the needles 105 disposed therein. For example, the shaft 202 may comprise a polymer and/or stainless steel braid.

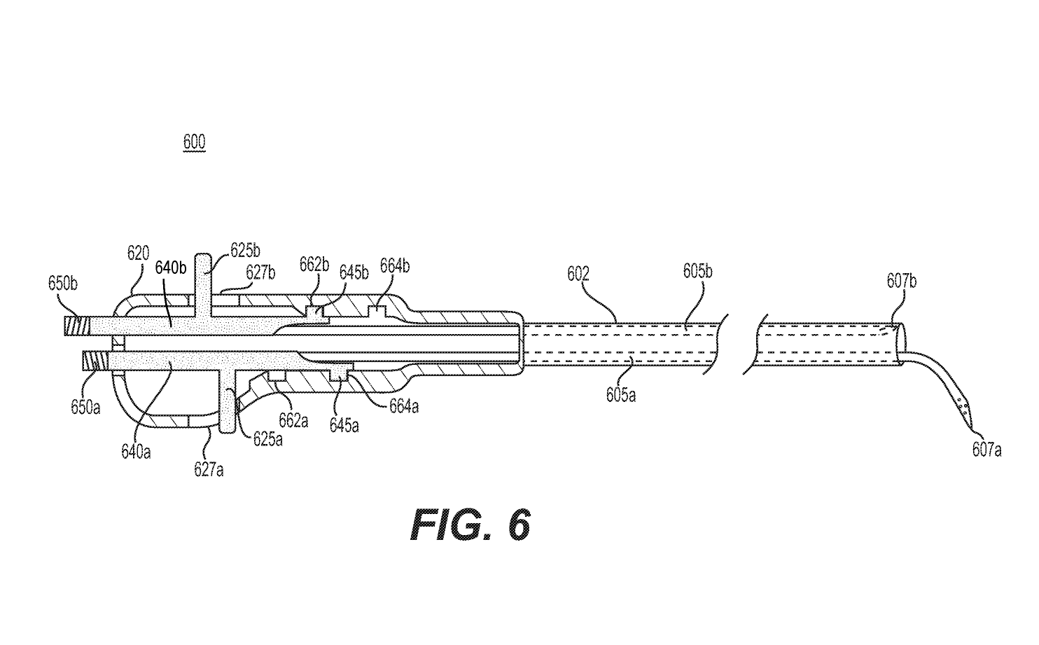

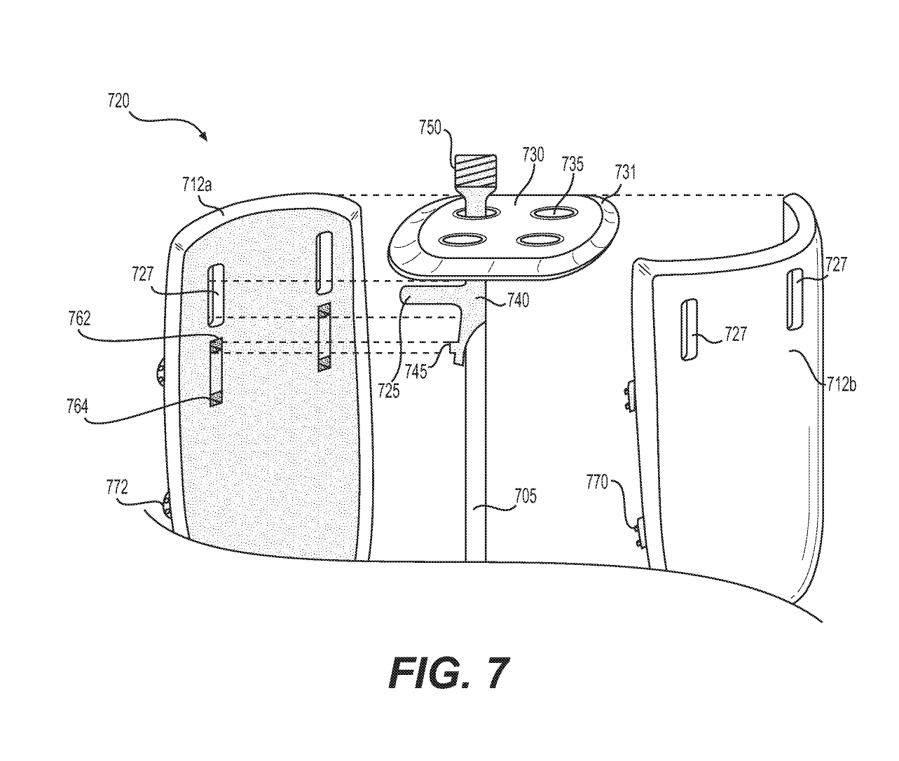

The handle 220 may include one or more actuators 225 for deploying the needles 105. In some embodiments, the handle 220 may include a separate actuator 225 for each needle 105 to allow control of the needles 105 independently of one another. For example, each actuator 225 may be configured to move a corresponding needle 105 longitudinally within the shaft 202 from a retracted position (wherein the needle tip 107 is disposed within the device 200) to an extended, deployed position (wherein the needle tip 107 extends past the distal end of the device 200). The actuators 225 may be directly coupled to proximal portions of the needles 105 (see FIGS. 6 and 7, below).

Each actuator 225 may be configured to move within a slot 227 of the handle 220, as shown in FIG. 2. For example, when the actuator 225 is in a distal position, the corresponding needle 105 may extend through the end cap 204, beyond the distal-most end of the device 200. Moving the actuator 225 proximally within the slot 227 may retract the needle 105 within the device 200, such that the tip 107 of each needle 105 does not extend past the end cap 204. Each slot 227 thereby may provide proximal and distal stops for respectively limiting the retraction and extension of the corresponding needle 105. Other features of the handle 220 described herein may provide alternative or additional stops.

Each needle 105 may define a lumen for the passage of fluid therethrough, e.g., supplied from a fluid reservoir connectable to the device 200. For example, the lumen of each needle 105 may be in communication with a port 250 coupled to a proximal end 230 of the handle 220. FIG. 2 shows four separate ports 250, each corresponding to a different needle 105. In some embodiments, however, each needle 105 may be in communication with a single port 250. Each port 250 may include a luer-type connection for receiving a fluid reservoir, such as a syringe 270. The syringes 270 may include sequential markings to quantify the dosage of fluid being injected, and may be disconnected from the device 200 as needed for replenishing fluid and/or changing the type of fluid for injection. In some embodiments, the device 200 may be configured to inject tissue with two or more different types of fluid simultaneously, from different fluid reservoirs and via different needles 105. In some embodiments, the device 200 may be configured to inject tissue with fluid delivered from each needle 105 simultaneously (e.g., from different fluid reservoirs or a single fluid reservoir coupled to each needle 105).

The needles 105 may be configured to deflect radially outward (e.g., towards body tissue) upon exiting the end cap 204. When retracted within the device 200, the distal portion of each needle 105 may extend substantially parallel to the longitudinal axis of the device 200, and when deployed, the distal portion of each needle 105 may deflect radially outward. The deflection angle of each needle 105 in the extended configuration may be the same or different from the deflection angle of any other needle 105. For example, one or more of the needles 105 may be deflected at a smaller or greater angle (with respect to the longitudinal axis of the device) than the other needles 105. According to some aspects, one or more of the needles 105 may exit the end cap 204 without deflecting, e.g., remaining substantially parallel to the longitudinal axis of the device 200.

The needles 105 may comprise any suitable material with sufficient flexibility for deflection (e.g., for any needles 105 being deflected) and rigidity for puncturing tissue, including, but not limited to, metals, metal alloys, and polymers. In some embodiments, for example, the needles 105 may comprise stainless steel or a shape memory material such as Nitinol that allows the distal portion of each needle 105 to assume a pre-set deflected configuration once unconstrained by the end cap 204 and/or shaft 202. Further, the needles 105 may have the same length or different lengths. For example, one of the needles 105 may be relatively longer than the others to allow the needle 105 to extend farther past the distal end of the device 200 and into tissue.

The end cap 204, shaft 202, and/or needles 105 may be configured for fluoroscopic and/or ultrasound guidance during a medical procedure. For example, one or more needles 105 may include an echogenic pattern visible via ultrasound and/or radiopaque marker visible via fluoroscopy to assist in positioning the needles 105 with respect to target tissue. In addition or alternatively, the end cap 204 and/or shaft 202 may include a radiopaque marker or echogenic pattern to visualize the distal end of the device 200 in relation to a tissue surface.

Further, each needle 105 may have a distinctive visual marking to assist a user in delivering fluid to a targeted area via particular needles 105. For example, a first needle 105 may have a single radiopaque stripe on a distal portion of the needle (e.g., proximate the tissue to be treated) that matches a single radiopaque stripe on the actuator 225 and/or port 250 corresponding to that first needle 105. Similarly, a second needle 105 may have two radiopaque stripes on a distal portion of the needle that matches two radiopaque stripes on the actuator 225 and/or port 250 corresponding to the second needle 105. Thus, the distinct markings may allow the user to identify which actuator 225 and/or port 250 should be accessed to actuate a particular needle 105.

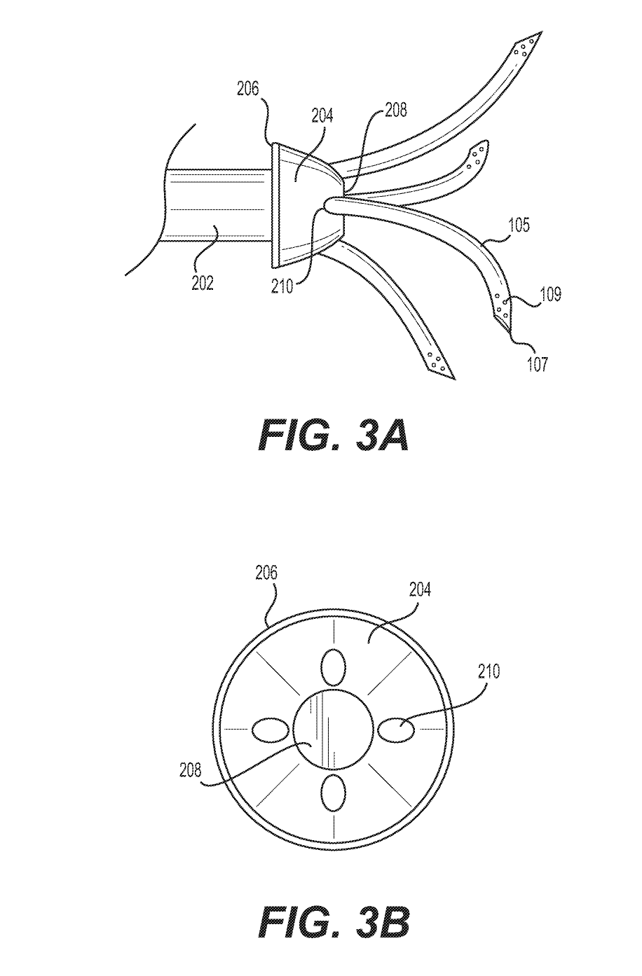

The end cap 204 may include one or more openings for the needles 105 to pass therethrough. FIG. 3A shows a side view of the distal end of the device 200 of FIG. 2 (including the shaft 202, the end cap 204, and four needles 105 extending through the end cap 204), and FIG. 3B shows an end view of FIG. 3A without the needles 105 for clarity. As shown, the end cap 204 may include a separate opening 210 corresponding to each needle 105. The openings 210 may be disposed about the circumference of the end cap 204, between the proximal end 206 and distal end 208 of the end cap 204 (e.g., proximate the distal end 208 as shown). The openings 210 may be symmetrically arranged, e.g., approximately 90 degrees apart for four needles 105 (see FIG. 3B), about 120 degrees apart for three needles 105, about 72 degrees apart for five needles 105, etc. According to some aspects, at least one of the needles 105 may exit the end cap 204 without being deflected, e.g., having a straight configuration substantially parallel to the longitudinal axis of the device 200, and the distal end 208 of the end cap 204 may define a corresponding opening for the passage of each of the needles therethrough. As mentioned above in connection to device 100, the end cap 204 may be configured to center the device 200 within a body lumen, e.g., having an outermost cross-sectional area at the proximal end 206 approximately the same as the diameter of the body lumen. In some embodiments, the end cap 204 may include an opening extending from the proximal end 206 to the distal end 208 of the end cap 204 and in communication with the lumen of the shaft 202 for receiving a guidewire, fluid, or gas.

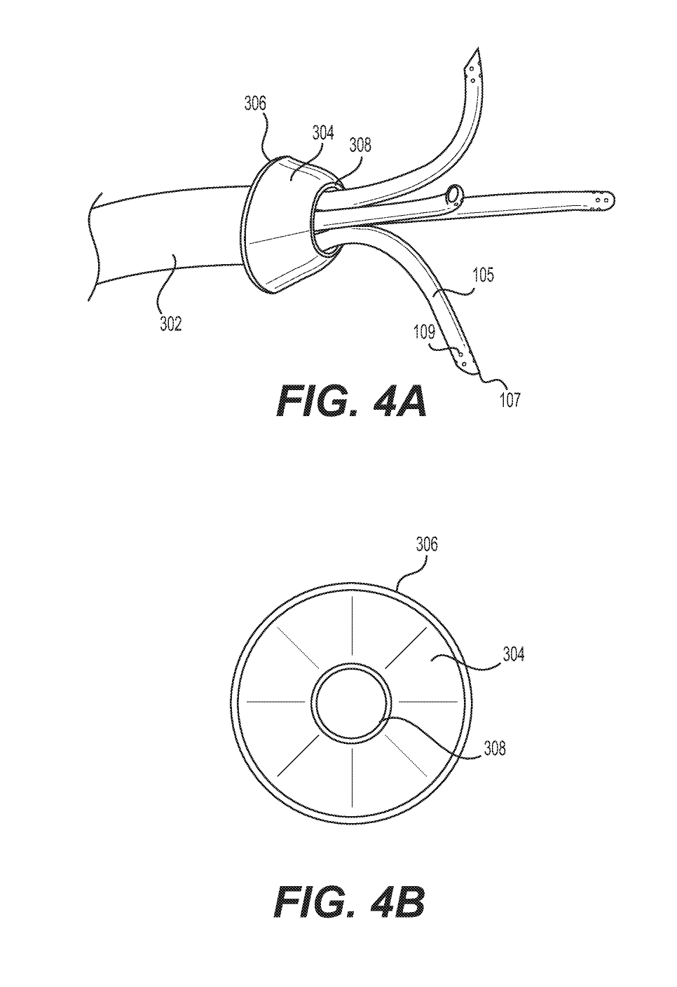

FIGS. 4A and 4B illustrate another end cap 304 (which may have any of the features of end caps 104 and/or 204 discussed above) suitable for the devices and methods disclosed herein. FIG. 4A shows a distal portion of a device including a shaft 302 coupled to the end cap 304, and four needles 105 extending through the end cap 304. FIG. 4B shows an end view of FIG. 4A without the needles 105 for clarity. As shown, the end cap 304 extends from a proximal end 306 to a distal end 308 in a generally frustoconical shape. Rather than having separate openings for each needle 105, the distal end 308 defines a single opening for the passage of all of the needles 105. Embodiments combining various features of end caps 204 and 304 are further contemplated herein, e.g., comprising an opening configured for the passage of two or more needles 105 and one or more additional openings configured for the passage of only one needle 105.

In embodiments comprising an end cap and a shaft as separate components, the end cap and the shaft may be coupled together via any suitable mechanism. FIGS. 5A, 5B, and 5C illustrate various mechanisms for attaching an end cap to a shaft (e.g., via complementary mating elements), which may be used in any of the embodiments disclosed herein.

FIG. 5A shows a shaft 502 and an end cap 504, each having a threaded portion (e.g., complementary mating elements) for screwing the end cap 504 to the shaft 502. The shaft 502 includes a plurality of needles 105 therein, e.g., disposed within a single lumen of the shaft 502. As shown, the shaft 502 may include an outer threaded portion 511 (first mating element) proximate the distal end 503 of the shaft 502 complementary to an inner threaded portion 513 (second mating element) of the end cap 504. For example, the proximal end 506 of the end cap 504 may define a lumen 512 having threads 513 along an inner surface of the lumen 512. The threaded portions 511, 513 may allow for the end cap 504 to be selectively attached and detached from the shaft 502.

The end cap 504 may include a plurality of openings 510 each allowing for the passage of one of the needles 105 therethrough. Each opening 510 may be in communication with the lumen 512 (and in communication with the inner lumen of the shaft 502), such that the needles may be extended and withdrawn into the end cap 504. In some embodiments, the distal end 508 of the end cap 504 may define an opening as an alternative to, or in addition to, the openings 510 along the length of the end cap 504.

The shaft 502 and/or the end cap 504 may include features to assist in aligning the needles 105 with the openings in the end cap 504. For example, the end cap 504 may define a single proximal opening that branches out along the length of the end cap 504 into separate openings, one opening corresponding to each needle 105 (e.g., four openings shown in FIG. 5A). The needles 105 may be actuated separately to slide into each of the openings as needed. According to some aspects, each opening may be only slightly larger than the outer diameter of a corresponding needle 105, such that only one needle 105 at a time may pass through each opening.

The threaded portions 511, 513 may be configured to align the needles 105 with the openings when the end cap 504 is fully screwed onto the shaft 502 (or alternatively when the shaft 502 is fully screwed onto the end cap 504). For example, translation of each needle 105 along the length of the shaft 502 may be confined to a pre-determined path to allow the position of the end cap 504 to be adjusted relative to the shaft 502 for aligning the openings with the needles 105. According to some aspects, the shaft 502 may include a separate lumen for receiving each needle 105. Additionally or alternatively, the needle 105 may include a proximal actuator confined within a slot of the handle (e.g., actuator 225 confined within slot 227 of handle 220 in FIG. 2) to confine translation of the needle 105 along a pre-determined path.

According to some aspects, the end cap 504 and the shaft 502 may include additional mating features to align the openings with the needles 105 once the end cap 504 and the shaft 502 are at least partially connected via the threaded portions 511, 513. For example, the end cap 504 and the shaft 502 may include any of the pairs of mating features of end cap 524 and shaft 522 of FIG. 5B, and/or of end cap 544 and shaft 542 of FIG. 5C discussed below, wherein the end cap 504 and the shaft 502 may be threaded together to engage the pairs of mating elements when the openings and needles 105 are aligned.

FIG. 5B shows a shaft 522 and an end cap 524 having complementary mating elements for securing the end cap 524 to the shaft 522. As in FIG. 5A, the shaft 522 may include a plurality of needles 105 therein, e.g., disposed within a single lumen of the shaft 502. The shaft 522 may include a pair of projections 531 (first mating elements) disposed on opposite sides of the shaft 522 (180 degrees apart) proximate the distal end 523 of the shaft 522, each projection 531 including a slot 531a. The shaft 522 may include only one projection 531, or more than two projections 531, such as 3, 4, 5 or more projections 531.

The end cap 524 may include a pair of inwardly-facing extensions 533 (second mating elements) complementary to the projections 531 of the shaft 522, and configured to fit within the slots 531a of the projections 531. The end cap 524 may have a number of extensions 533 corresponding to the number of projections 531, e.g., only one extension 533, or 3, 4, 5, or more extensions 533. As the end cap 524 is pressed down on the distal end 523 of the shaft 522, the extensions 533 may pivot outward about a point 534 at the proximal end 526 of the end cap 524 as they contact the distal-most portion of the projections 531. Once the extensions 533 reach the slots 531a, they may snap into the slots 531a to secure the end cap 524 to the shaft 522. The end cap 524 may be removed by pulling distally with enough force to pull the extensions 533 out of the slots 531a.

In some embodiments, the shaft 522 may include slots (first mating elements) rather than projections 531 proximate the distal end 523 of the shaft 522, the slots configured to receive the extensions 533 (second mating elements) of the end cap 524. For example, the extensions 533 may be biased radially inward, such that pressing the end cap 524 onto the shaft 522 may cause the extensions 533 to pivot outward, about the proximal end 526 of the end cap 524. Upon reaching the slots, the extensions 533 may snap into the slots to secure the end cap 524 to the shaft 522. The end cap 524 may be removed by pulling distally with enough force to pull the extensions 533 out of the slots within the shaft 522.

The proximal end 526 of the end cap 524 may define a lumen 532 to receive the distal end 523 of the shaft 522 and in communication with a plurality of openings 530 for the passage of needles 105 therethrough, similar to end cap 504 of FIG. 5A. The distal end 528 of the end cap 524 may be closed (as shown), or may define an opening as an alternative to, or in addition to, the openings 530 along the length of the end cap 524. The shaft 522 and/or the end cap 524 may include features to assist in aligning the needles 105 with the openings in the end cap 524, including any of the features discussed above in connection to shaft 502 and end cap 504. According to some aspects, the shaft 522 and the end cap 524 may have complementary cross-sectional areas that allow the shaft 522 and the end cap 524 to be connected with only one orientation relative to each other. For example, each of the shaft 522 and the end cap 524 may be D-shaped, or any other suitable cross-sectional shape for aligning the holes of the end cap 524 to the needles 105 within the shaft 522.

FIG. 5C shows a shaft 542 and an end cap 544 having complementary mating elements for securing the end cap 544 to the shaft 542. As in FIGS. 5A and 5B, the shaft 542 may include a plurality of needles 105 therein, e.g., disposed within a single lumen of the shaft 542. The shaft 542 may include a pair of projections 551 (first mating elements) disposed on opposite sides of the shaft 522 (180 degrees apart) proximate the distal end 543 of the shaft 542. Each projection 551 may define a cavity with a distally-facing open end. The shaft 542 may include only one projection 551, or more than two projections 551, such as 3, 4, 5 or more projections 551.

The end cap 544 may include a pair of outward-facing elements 553 (second mating elements) with a shape complementary to the projections 551 of the shaft 542, e.g., configured to fit within the cavity defined by the projections 551. The end cap 544 may have a number of extensions 553 corresponding to the number of projections 551, e.g., only one extension 553, or 3, 4, 5, or more extensions 553. As the end cap 544 is pressed down on the distal end 543 of the shaft 542, each extension 553 may press the distal-most lip portion 551a of a corresponding projection 551 radially outward until the widest portion of the extension 553 clears the lip portion 551a. The lip portion 551a then may snap around the extension 553, thus securing the end cap 544 to the shaft 542. The end cap 544 may be removed by pulling distally with enough force to pull the extensions 553 past the lip portions 551a of the projections 551.

The proximal end 546 of the end cap 544 may define a lumen 552 to receive the distal end 553 of the shaft 542 and in communication with a plurality of openings 550 for the passage of needles 105 therethrough, similar to end caps 504 and 524 discussed above. The distal end 548 of the end cap 544 may be closed (as shown), or may define an opening as an alternative to, or in addition to, the openings 550 along the length of the end cap 544. The shaft 542 and/or the end cap 544 may include features to assist in aligning the needles 105 with the openings in the end cap 544, including any of the features discussed above in connection to shaft 502 and end cap 504 and/or in connection to shaft 522 and end cap 524.

An exemplary mechanism for deploying needles into tissue is shown in FIG. 6 for a device 600, which may include any of the features of devices 100 and/or 200 discussed above. FIG. 6 shows a cut-away view of the device 600, including a handle 620, a shaft 602, and two needles 605a, 605b extending generally parallel to each other through the handle 620 and the shaft 602. While not shown, the device 600 may include an end cap having any features of the end caps 104, 204, 304, 504, 524, and/or 544 discussed above.

One of the needles 605a is depicted in an extended, deployed configuration, wherein the distal tip 607a of the needle 605a extends past the distal end of the shaft 602 and deflects radially outward. The other needle 605b is depicted in a retracted configuration, wherein the distal tip 607b is retained within the shaft 602. Each needle 605a, 605b may include any of the features of needles 105 discussed above (e.g., sharp distal tips 607a, 607b, one or more side apertures, etc.). While two needles 605a, 605b are shown, the device 600 may include only one needle (e.g., needle 605a) or 3, 4, or 5 or more needles. The needles 605a, 605b may comprise any suitable material, including metals, metal alloys, polymers, and shape member materials as mentioned above regarding needles 105 of device 100.

Each needle 605a, 605b may define a lumen for the passage of fluid therethrough. Referring to one of the needles 605a as an example (the following features being equally applicable to the other needle 605b), the needle 605a may define a lumen from the proximal end 650a to the distal tip 607a. The needle 605a may include an outer member 640a, e.g., configured as a sleeve surrounding only a portion of the needle 605a, e.g., a proximal portion as shown in FIG. 6. For example, the outer member 640a may extend from the proximal end 650a to a middle portion of the needle 605a disposed within the handle 620. The proximal end 650a may include a luer-type connection or any other suitable attachment for coupling to a fluid reservoir. For example, the proximal end 650a may be configured to receive a syringe 270 as discussed above regarding device 200 shown in FIG. 2. A fluid may be injected via the proximal end 650a of the needle 605a and flow distally through the needle lumen to exit at, or proximate to, the tip 607a.

In some embodiments, the outer member 640a may include a radial protrusion 625a extending through a slot 627a of the handle 620 to act as an actuator. For example, a user may move the actuator 625a proximally and distally within the slot 627a to control longitudinal movement of the needle 605a. Similar to device 200 discussed above, moving the actuator 625a proximally may withdraw the tip 607a within the shaft 602 (and/or within an end cap at the distal end of the shaft 602), and moving the actuator 625a distally may extend the tip 607a beyond the distal end of the shaft 602 (as shown in FIG. 6), allowing the distal portion of the needle 605a to contact tissue.

The device 600 may include a locking mechanism to secure the position of the first needle 605a with respect to the handle 620 and/or shaft 602. In some embodiments, for example, the outer member 640a may include one or more pins 645a having a shape complementary to one or more depressions 664a along the inner surface of the handle 620. FIG. 6 illustrates an example wherein the outer member 640a comprises one pin 645a (e.g., distal to the actuator 625a) having a generally rectangular cross-section complementary to each of two depressions 662a, 664a aligned along the inner surface of the handle 620. As the first needle 605a translates along the longitudinal axis of the device 600, the pin 645a may engage with either of the depressions 662a, 664a. To lock the first needle 605a in a retracted configuration, for example, a user may slide the actuator 625a proximally until the pin 645a engages the first (proximal) depression 662a. The outer member 640a may be configured such that the pin 645a is biased radially outward, yet able to flex radially inward upon application of force. Thus, upon reaching the depression 662a, the pin 645a may snap into engagement with the depression 662a, thereby preventing further movement of the first needle 605a.

To deploy the needle 605a for injecting fluid into tissue, the user may apply sufficient force to the actuator 625a to disengage the pin 645a from the first depression 662a, thus allowing the needle 605a to move distally. Once the pin 645a reaches the second (distal) depression 664a, corresponding to an extended configuration of the needle 605a as shown in FIG. 6, the pin 645a may snap into engagement with the second depression 664a to prevent further movement of the needle 605a with respect to the handle 620 and/or shaft 602. While FIG. 6 illustrates two locking positions for each needle 605a, 605b (e.g., retracted and extended configurations), the device 600 may include any number and combination of pins and depressions to secure the needles in desired positions.

As mentioned above, additional needles of the device 600 may include all or some of the same features of needle 605a. Thus, another needle 605b as shown in FIG. 6 extends from a proximal end 650b to a distal tip 607b. The proximal end 650b of the second needle 605b may include the same or different type of connection with respect to the first needle 605a for coupling to a fluid reservoir. Further, the needle 605b may include an outer member 640b comprising a radial protrusion/actuator 625b movable within a corresponding slot 627b of the handle 620. The outer member 640b may include one or more pins 645b configured to engage one or more corresponding depressions 662b, 664b along the inner surface of the handle 620.

The needles 605a, 605b may be independently actuated (e.g., via separate actuators 625a, 625b), thus allowing a user to manipulate and position the needles 605a, 605b according to the particular anatomy of the patient. For example, the patient may have a larger mass of tissue to one side of the esophagus, requiring one of the needles (e.g., needle 605a) to be extended farther distally to inject fluid more deeply into the tissue. Further, by coupling the needles 605a, 605b to separate fluid reservoirs (e.g., separate syringes 270), the user may adjust the type of fluid and/or fluid dosage being injected at a particular tissue site.

According to some aspects of the present disclosure, the locking mechanism may define a proximal-most stop (e.g., depressions 662a, 662b) and a distal-most stop (e.g., depressions 664a, 664b) that limit the extension length of the needles 605a, 605b. Devices according to the present disclosure need not include a locking mechanism, however. According to some aspects of the present disclosure, the length of the slots 627a, 627b (and hence the translation length of the actuators 625a, 625b) may determine the maximum length the needles 605a, 605b may extend beyond the distal end of the shaft 602. The slots 627a, 627b therefore may have dimensions that allow the needles 605a, 605b to reach their full extension necessary to perform a medical procedure.

In some embodiments, the handle of the device may include two or more pieces attached together to form the body of the handle. FIG. 7 shows an exploded view of an exemplary handle 720, which may include any of the features of handles 220 and/or 620 discussed above, the handle 720 including two opposing body pieces 712a, 712b. The two body pieces 712a, 712b may have complementary attachment features, e.g., snap-fit pins 770 insertable into holes 772 as shown, for attaching the pieces 712a, 712b together. The handle 720 also may include a separate end piece 730 to form a proximal end of the handle 720. The term “end piece” is used herein for convenience in referring to the figures, and does not exclude devices according to the present disclosure that may have features proximal to the end piece. The end piece 730 may connect to the body pieces 712a, 712b via any suitable mechanism. For example, the end piece 730 may include ridges marking an outer rim 731 of the end piece 730 to align each body piece 712a, 712b with the end piece 730. The dimensions of the body pieces 712a, 712b and the end piece 730 may be complementary to one another as mating elements. According to some aspects, the body pieces 712a, 712b and the end piece 730 may be attached together via adhesive, ultrasonic welding, snap-fits, or other manufacturing methods for coupling different components together.

The end piece 730 may serve as a needle holder, e.g., defining one or more openings 735 for receiving a corresponding number of needles 705. According to some aspects, one of the body pieces 712a, 712b may be integral with the end piece 730, or each body piece 712, 712b may be integral with a portion (e.g., one half) of the end piece 730, such that one or both of the body pieces 712a, 712b may include proximal openings 735 for receiving the needles 705. As shown in FIG. 7, the end piece 730 includes four openings 735 to receive four needles 705, only one needle 705 depicted for clarity. The needle 705 may include any of the features of needles 105 and/or 605a, 605b discussed above. For example, the proximal end 750 of the needle 705 extending outside the handle 720 may include a luer-type connection (or other suitable type of connection) for receiving a syringe or other fluid reservoir. The proximal end 750 may comprise part of an outer member 740 including a protrusion/actuator 725 configured to extend through a complementary slot 727 of the handle 720 (e.g., a slot 727 defined by body piece 712a). The outer member 740 also may include a pin 745 configured to fit within complementary depressions 762, 764 within the wall of the handle 720 (e.g., depressions 762, 764 within the inner wall of body piece 712a).

As mentioned above, the devices disclosed herein may be useful in various medical procedures, e.g., for injecting a fluid into tissue along an anatomic wall. Referring once again to FIG. 1, a physician or medical technician may insert a device 100 (or device 200 or 600 discussed above) into the esophagus 10 or other body lumen in need of treatment. The proximal end 106 of the end cap 104 may have a cross-sectional dimension approximately equal to (e.g., on the order of) the diameter of the esophagus 10 to center the device 100 within the esophagus 10 without the need for an endoscope or active intervention by the physician. The device 100 may be advanced through the esophagus 10 until reaching a stricture or narrowed region caused by abnormal tissue 15.

The physician may deploy needles 105 into the tissue 15 via proximal actuators as discussed above (e.g., actuators 625a, 625b of device 600), such that the needles 105 puncture the tissue 15. The needles 105 may be deployed simultaneously (e.g., by manipulating the corresponding needle actuators simultaneously) or independently (e.g., by manipulating the corresponding actuators separately, such as in sequence), and may be extended different lengths past the distal end 108 of the end cap 104.

A fluid such as ethanol may be injected through each needle 105 before, during, or after puncturing the tissue 15. For example, the physician may choose first to deploy the needles 105 into the tissue 15, and then to inject ethanol into the tissue 15, e.g., by pushing down the plunger of a syringe coupled to each needle 105. The type of fluid and dosage of fluid delivered to the tissue 15 through each needle 105 may be adjusted according to the needs of the patient, e.g., based on the characteristics and amount of tissue 15. Shrinkage in the tissue 15 may be observed within a day or several days (e.g., about 2-4 days) of the ethanol injection, increasing the patient's ability to ingest food and liquids through the esophagus 10.

Exemplary fluids that may be injected include, but are not limited to, alcohols (e.g., ethanol, methanol, isopropanol), antiseptic agents, anesthetic agents, analgesic agents, chemotherapeutic agents, other pharmaceutical agents or drugs, saline, water, and any combinations thereof. The alcohols and other fluids may have any suitable purity. According to some aspects, for example, ethanol having a purity greater than about 90%, such as ranging from about 95% to about 100%, e.g., a purity greater than about 99% (absolute ethanol) may be injected into tissue via the devices and methods disclosed herein. The dosage per injection may range from about 0.1 cc (cubic centimeters) to about 10 cc of fluid, e.g., aliquots of about 1 cc, 2 cc, 5 cc, 7 cc, or 10 cc. Based on the patient response (e.g., shrinkage of tissue 15), the physician may repeat the treatments according to a prescribed regimen.

Other embodiments of the present disclosure will be apparent to those skilled in the art from consideration of the specification and practice of the embodiments disclosed herein. While certain features of the present disclosure are discussed within the context of exemplary procedures (e.g., fluid injection into esophageal tissue), the devices and methods described herein may be used in other areas of the body, and for other medical procedures according to the general principles disclosed. The features of any embodiment may be used in combination with any other embodiment.

It is intended that the specification and examples be considered as exemplary only, with a true scope and spirit of the present disclosure being indicated by the following claims.

We claim:

1. A medical device comprising:
a handle including a plurality of actuators;
a shaft; and
a plurality of needles extending through the shaft, each needle defining a lumen, and each needle corresponding to one of the plurality of actuators for movement along a longitudinal axis of the device from a retracted configuration to an extended configuration, independent of movement of the other needles of the plurality of needles;
wherein, in the extended configuration, a distal portion of at least one of the needles deflects radially outward with respect to the longitudinal axis of the device.

2. The medical device of claim 1, wherein each actuator extends through a corresponding slot of the handle.

3. The medical device of claim 1, wherein the distal portion of each needle includes a sharp tip configured to puncture tissue.

4. The medical device of claim 1, wherein the distal portion of at least one of the needles includes a plurality of apertures disposed circumferentially around a wall of the needle.

5. The medical device of claim 1, wherein a proximal portion of each needle includes an attachment for coupling the needle to a fluid reservoir.

6. The medical device of claim 5, wherein the fluid reservoir comprises a syringe.

7. The medical device of claim 1, wherein the device includes an actuator for controlling a supply of fluid to at least one of the needles.

8. The medical device of claim 7, wherein the actuator for controlling the supply of fluid is separate from the actuators that control movement of the needles along the longitudinal axis of the device.

9. The medical device of claim 1, further comprising an end cap coupled to a distal end of the shaft, the end cap including at least one opening for the passage of one or more of the needles distally through the end cap.

10. The medical device of claim 9, wherein the end cap includes a plurality of openings arranged circumferentially about the end cap at a same radial position between a proximal end of the end cap and a distal end of the end cap, each needle being aligned with a corresponding one of the openings.

11. The medical device of claim 10, wherein the end cap defines a lumen in communication with each opening of the plurality of openings.

12. The medical device of claim 9, wherein the end cap is detachable from the shaft.

13. The medical device of claim 1, wherein the handle includes a locking mechanism corresponding to each needle of the plurality of needles to selectively lock the corresponding needle in at least one of the retracted configuration or the extended configuration.

14. A medical device comprising:
a handle;
a shaft;
an end cap coupled to a distal end of the shaft; and
a plurality of needles extending through the shaft and at least partially through the end cap, each needle defining a lumen and being independently moveable relative to other needles of the plurality of needles along a longitudinal axis of the device to transition from a retracted configuration to an extended configuration;
wherein, in the extended configuration, a distal portion of each needle deflects radially outward with respect to the longitudinal axis of the device.

15. The medical device of claim 14, wherein the end cap includes a plurality of openings arranged circumferentially about the end cap at a same radial position between a proximal end of the end cap and a distal end of the end cap, each needle being aligned with one of the openings.

16. The medical device of claim 14, wherein the end cap is detachable from the shaft.

17. The medical device of claim 14, wherein the end cap includes at least one mating element complementary to a mating element of the shaft for coupling the end cap to the shaft.

18. A medical device comprising:
a handle;
a shaft;
an end cap coupled to a distal end of the shaft;
a plurality of needles extending through each of the shaft and the end cap, each needle defining a lumen and being independently moveable relative to other needles of the plurality of needles along a longitudinal axis of the device to transition from a retracted configuration to an extended configuration; and
a locking mechanism corresponding to each needle of the plurality of needles to selectively lock the corresponding needle in at least one of the retracted configuration or the extended configuration.

19. The device of claim 18, wherein, in the extended configuration, a distal portion of each needle deflects radially outward with respect to the longitudinal axis of the device.

20. The device of claim 19, wherein the handle includes a plurality of actuators, each actuator configured to move a corresponding one of the needles along the longitudinal axis of the device independently of the other needles.
